(19) United States
(12) Patent Application Publication
(10) Pub. No.: US 2022/0226643 A1
(43) Pub. Date: Jul. 21, 2022

(54) S100 MODULATION

(71) Applicant: Leonhardt Ventures LLC, Mission Viejo, CA (US)

(72) Inventor: Howard J. Leonhardt, Mission Viejo, CA (US)

(21) Appl. No.: 17/571,332

(22) Filed: Jan. 7, 2022

Related U.S. Application Data
(60) Provisional application No. 63/139,706, filed on Jan. 20, 2021.
(60) Provisional application No. 63/219,597, filed on Jul. 8, 2021.

(51) Int. Cl. A61N 1/05; A61K 35/28; A61N 1/04

(52) U.S. Cl. CPC A61N 1/0592; A61N 1/0464; A61K 35/28

(57) ABSTRACT

Described is a low voltage, pulsed electrical stimulation device (bioelectric stimulator associated with electrodes) for controlling expression of S100 protein(s) by cellular tissues. The bioelectric stimulator is useful in methods to treat a subject suffering from bladder, heart, and/or nerve tissue damage.

### CROSS-REFERENCE TO RELATED APPLICATIONS

This application claims the benefit under 35 U.S.C. § 119(e) of U.S. Provisional Patent Application Ser. No. 63/139,706, filed Jan. 20, 2021, and U.S. Provisional Patent Application Ser. No. 63/219,597, filed Jul. 8, 2021, the disclosure of each of which is hereby incorporated herein in its entirety by this reference.

### TECHNICAL FIELD

The application relates generally to the field of medical devices and associated treatments, and more specifically to precise bioelectrical stimulation of a subject's tissue, possibly augmented with the administration of a composition comprising, among other things, stem cells and nutrients, useful to stimulate and treat the subject, the subject's tissue(s), the subject's organ(s), and/or the subject's cells. More specifically, the application relates to a device having programmed bioelectric signaling sequences, and associated methods for the controlled modulation of S100 via precise bioelectrical signaling sequences useful in, for example, reducing cardiac scar burden, muscle treatment and regeneration, and improving contractile function of the heart.

### BACKGROUND

Winters et al. (2016) infra, described the development of various strategies for regenerative medicine in an attempt to offer a therapeutic option for the repair and potential regeneration of damaged cardiac tissue post-myocardial infarction (“MI”). Human umbilical cord sub-epithelial cell-derived stem cells (“hUC-SECs”), human bone marrow-derived mesenchymal stem cells (“hBM-MSCs”), and human induced pluripotent stem cell-derived cardiomyocytes (“hiPSC-CMs”), all derived from human tissue, were shown to have in vitro and in vivo therapeutic potential. Additionally, S100a1, VEGF165, and stromal-derived factor-1a (SDF-1a) genes all have the potential to improve cardiac function and/or effect adverse remodeling. Winters et al. compared the therapeutic potential of hBM-MSCs, hUCSECs, and hiPSC-CMs along with plasmid-based genes to evaluate the in vivo potential of intramyocardially injected biologics to enhance cardiac function in a mouse MI model. Human cells derived from various tissue types were expanded under hypoxic conditions and injected intramyocardially into mice that had undergone left anterior descending (“LAD”) artery ligation. Similarly, plasmids were also injected into three groups of mice after LAD ligation. Seven experimental groups were studied in total: (1) control (saline), (2) hBM-MSCs, (3) hiPSC-CMs, (4) hUC-SECs, (5) S100a1 plasmid, (6) VEGF165 plasmid, and (7) SDF-1 a plasmid. Winters et al. evaluated echocardiography, hemodynamic catheterization measurements, and histology at 4 and 12 weeks post-biologic injection. Significant improvement was observed in cardiac function and contractility in hiPSC-CM and S100a1 groups and a significant reduction in left ventricle scar within the hUC-SEC group and a slight improvement in the SDF-1 a and VEGF165 groups compared to the control group. Winters et al. demonstrated the potential for the described biologic therapies to reduce scar burden and improve contractile function.

As described in Xia et al. “S100 Proteins As an Important Regulator of Macrophage Inflammation” Front. Immunol., (Jan. 5, 2018), the S100 proteins, are a family of calcium-binding cytosolic proteins, have a broad range of intracellular and extracellular functions through regulating calcium balance, cell apoptosis, migration, proliferation, differentiation, energy metabolism, and inflammation. The intracellular functions of S100 proteins involve interaction with intracellular receptors, membrane protein recruitment/transportation, transcriptional regulation and integrating with enzymes or nucleic acids, and DNA repair. The S100 proteins could also be released from the cytoplasm, induced by tissue/cell damage and cellular stress. The extracellular S100 proteins, serving as a danger signal, are crucial in regulating immune homeostasis, post-traumatic injury, and inflammation. Extracellular S100 proteins are also considered biomarkers for some specific diseases. In this review, we will discuss the multi-functional roles of S100 proteins, especially their potential roles associated with cell migration, differentiation, tissue repair, and inflammation.

S100A1 regulates both cardiac performance and vascular biology. In cardiomyocytes, S100A1, is known to substantially improve Ca′ handling and contractile performance. Additionally, S100A1 targets the cardiac sarcomere and mitochondria, leading to reduced pre-contractile tension as well as enhanced oxidative energy generation. S100A1 increasing endothelial nitric oxide synthase activity of endothelial cells. Myocardial infarction in S100A1 knockout mice resulted in accelerated transition towards heart failure and excessive mortality in comparison with wild-type controls. Mice lacking S100A1 furthermore displayed significantly elevated blood pressure values with abrogated responsiveness to bradykinin. Further, numerous studies in small and large animal heart failure models show that S100A1 overexpression results superior survival in response to myocardial infarction, indicating the high potential of S100A1-based therapeutic interventions.

### BRIEF SUMMARY

Described is a bioelectric stimulator programmed to produce at least one bioelectric signal that modulates (upregulates or downregulates) the expression of S100 in a mammalian target tissue. In certain preferred embodiments, the bioelectric stimulator upregulates the expression of S100 in the target tissue. Expression may be measured using, for example, real-time polymerase chain reaction or quantitative reverse transcription PCR. In certain embodiments, the bioelectric stimulator is actuated and runs through programmed signals to modulate the production of, e.g., S100 protein(s) such as S100a and/or S100A1. Preferably, the bioelectric signal is applied to the target cellular tissue for at least 30 minutes.

In certain embodiments, the described bioelectric stimulator produces at least one bioelectric signal selected from the group consisting of 10 Hz square, biphasic waveform at 50% duty, 75 Hz square, biphasic waveform at 50% duty, 250 Hz square, biphasic waveform at 50% duty, 500 Hz square, biphasic waveform at 50% duty, and a combination of any thereof.

In certain embodiments, the described bioelectric stimulator produces at least one bioelectric signal of 10 Hz square, biphasic waveform at 50% duty.

In certain embodiments, the described bioelectric stimulator produces at least one bioelectric signal of 75 Hz square, biphasic waveform at 50% duty.

In certain embodiments, the described bioelectric stimulator produces at least one bioelectric signal of about (plus or minus 5%) 250 Hz square, biphasic waveform at 50% duty.

In certain embodiments, the described bioelectric stimulator produces at least one bioelectric signal of about (plus or minus 5%) 500 Hz square, biphasic waveform at 50% duty.

In certain preferred embodiments, the at least one bioelectric signal produced by the bioelectric stimulator is from about (plus or minus 5%) 250 Hz square, biphasic waveform to about (plus or minus 5%) 500 Hz square, biphasic waveform.

A method of such bioelectric stimulators is to stimulate tissue of a subject, the method comprising: connecting the bioelectric stimulator to the target tissue of the subject, and actuating the bioelectric stimulator to produce the programmed bioelectric signal(s).

In certain embodiments, the described methods, devices, and therapy of S100A1 bioelectric protein expression are combined which other therapies including bioelectric expression of klotho, follistatin, insulin-like growth factor 1 (IGF-1), stromal cell-derived factor 1 (SDF1), platelet-derived growth factor (“PDGF”), and other bioelectric controlled regeneration promoting protein expressions, which may be combined with daily delivery via, for example, a catheter system delivering a mixed composition of muscle stem cells, bioelectric pre-treated PRF, secretome from amniotic sourcing and exosomes.

In certain embodiments, such a method may further include delivering to the tissue a composition comprising Human umbilical cord sub-epithelial cell-derived stem cells (“hUC-SECs”), human bone marrow-derived mesenchymal stem cells (“hBM-MSCs”), and human induced pluripotent stem cell-derived cardiomyocytes (“hiPSC-CMs”).

While not intending to be bound by theory, the described system utilizes precise bioelectric signal sequences that appear to communicate with DNA and cell membranes within stimulated tissues of the subject to cause the stimulated cells to upregulate expression of S100 protein(s) such as S100A1.

S100A1 contributes to improving heart contractile performance. Described herein is a bioelectric method to increase S100A1 delivery to hearts in need thereof instead of being limited to direct injections alone, making delivery more sustainable and practical for therapeutic strategies.

Useful indications include treatment of cardiac tissue, muscle treatment and/or regeneration, and use with patients post-myocardial infarction. The technology has potential application in bladder, heart, and nerve treatment and regeneration, including treatment of spinal cord injury.

### BRIEF DESCRIPTION OF THE DRAWINGS

FIG. 1 depicts a programmed bioelectric stimulator (with or without cell and growth factor) for delivery to the heart of a human subject via two lumens respectively at a silicon septum.

FIG. 2 depicts a programmed bioelectric stimulator depicted alongside a U.S. quarter.

FIG. 3 depicts an interface for use with the system.

FIG. 4 depicts a micropump for use with the system.

FIG. 5 depicts a pump associated with a subject's heart.

FIG. 6 is a bar graph depicting fold change in S100A expression due to treatment of cells at different frequencies.

### DETAILED DESCRIPTION

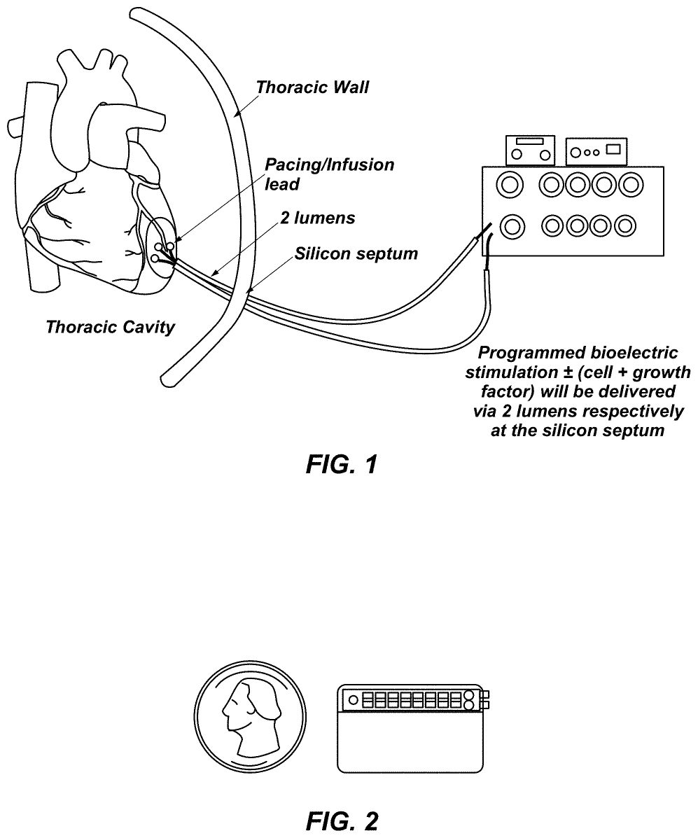

Referring now to FIG. 1, depicted is a human use bioelectric stimulator and infusion pump for use with treatment of, e.g., the heart. A bench top stimulator (e.g., a Mettler Model 240 Stimulator from Mettler Electronics of Anaheim, Calif., US) may be programmed with the described bioelectric signals. Preferably, such a device is about the size of two quarters (FIG. 2) and is programmable and re-fillable with low cell damage design. Refilling may be by silicon septum ports and reservoir chambers. Depicted particularly in FIG. 1 are the subject's heart, the pacing lead, the infusion lead, the thoracic cavity, two lumens, thoracic wall, silicon septum, and a larger programmed/programmable bioelectric stimulator, together with composition (e.g., cells and growth factors) for delivery via two lumens via the silica septum. A microinfusion pump for continuous or repeat delivery of a liquid composition, which microinfusion pump includes silicon septum ports and associated reservoir chambers connected to the bioelectric stimulator microinfusion pump to the tissue with a pacing infusion lead.

The underlying bioelectric stimulators can be obtained from OEM suppliers as well as their accompanying chargers and programmers. These electric signal generators are programmed to produce specific signals (“bioelectric signals”) that modulate specific protein expression at precisely the right time for, e.g., optimal treatment or regeneration.

The bioelectric stimulator preferably comprises an electric signal generator, which is programmable to produce the described bioelectric signals. A micro voltage signal generator for use herein may be produced utilizing the same techniques to produce a standard heart pacemaker well known to a person of ordinary skill in the art. An exemplary microvoltage generator is available from Mettler Electronics Corp. of Anaheim, Calif., US or HTM Electronica of Amparo, B R. The leading pacemaker manufacturers are Medtronic, Boston Scientific Guidant, Abbott St. Jude, BioTronik and Sorin Biomedica. In certain embodiments, the bioelectric signal is applied to the subject wirelessly or via wired electrode(s) or lead(s). See, e.g., the incorporated herein U.S. Pat. No. 10,960,206 to Leonhardt et al. (Mar. 30, 2021) for “Bioelectric Stimulator”.

The bioelectric stimulator may be produced and programmed utilizing techniques such as those used to produce a standard heart pacemaker, which is well known to a person of ordinary skill in the art. Microvoltage generators, which can be appropriately configured and programmed, are commercially available. The primary difference is the special electrical stimulation signals needed to modulate expression of S100. The leading pacemaker manufacturers include Medtronic, Boston Scientific Guidant, Abbott St. Jude, BioTronik and Sorin Biomedica.

Optimal results for S100A1 expression of bioelectric signal stimulation of porcine heart tissue (measured using RT-qPCR) were achieved with 30 minutes stimulation time using a square, biphasic waveform at 50%, 1.0V (as determined at the cellular level), and 75 Hz frequency where a greater than 275% increase in S100A1 expression over baseline was observed.

The bioelectric stimulator may be also programmed to generate further signals that modulate expression of other proteins such as, for example, klotho, LIM muscle, follistatin, stromal cell-derived factor 1 (“SDF1”), platelet-derived growth factor (“PDGF”), epidermal growth factor (“EGF”), IGF-1, and hepatocyte growth factor (“HGF”). (For exemplary such bioelectric signals, see, e.g., the incorporated U.S. Pat. No. 10,960,206 B2 to Leonhardt et al. and U.S. Patent Application Publication US 2020-0289826 A1 to Leonhardt et al. (Sep. 17, 2020) for “Klotho Modulation,” the contents of each of which are incorporated herein by this reference).

An infusion and electrode wide area pitch may be constructed by cutting conduction polymer to shape and forming plastic into a flat bag with outlet ports in strategic locations.

An organ specific matrix is a composition comprising cells of an organ which is to be treated. The organ specific matrix is believed to aid in stem cell differentiation, but in any event is found to be useful in the composition. It has been found that for the multicomponent composition, cells plus selected growth factors are better than just cells alone. See, e.g., Procházka et al. “Therapeutic Potential of Adipose-Derived Therapeutic Factor Concentrate for Treating Critical Limb Ischemia,” Cell Transplantation, 25(9), pp. 1623-1633(11) (2016) and “Cocktail of Factors from Fat-derived Stem Cells Shows Promise for Critical Limb Ischemia,” world wide web at sciencenewsline.com/news/2016012204520017.html (Jan. 22, 2016), the contents of each of which are incorporated herein by this reference.

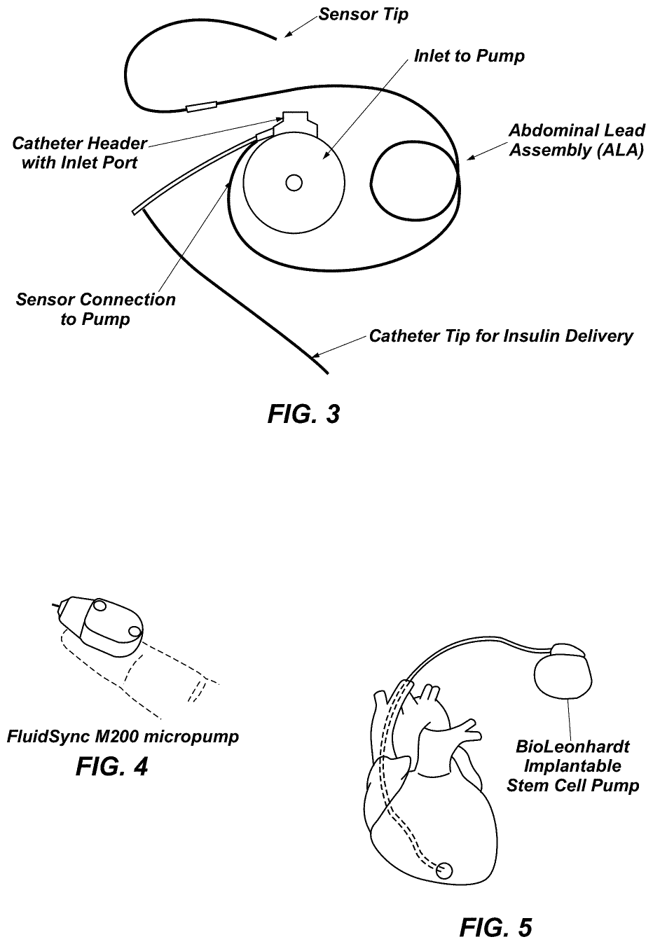

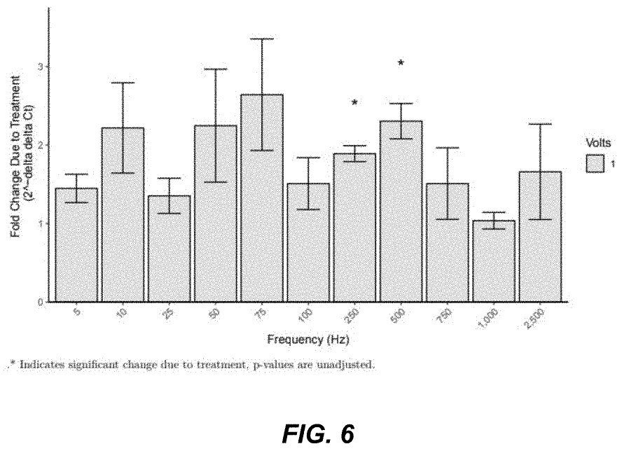

In case of an advanced disease state, a micro infusion pump (e.g., FIGS. 3-5) is used for daily delivery of, e.g., 2 ml of organ regeneration composition (comprised of adipose-derived cells or bone marrow-derived mesenchymal stem cells plus cocktail of growth factors (usually derived from amniotic fluid or placenta), selected Micro RNAs, selected alkaloids, selected anti-inflammatory agents, nutrient hydrogel, organ specific matrix, selected exosomes). For muscle regeneration, immature myoblasts are included in the composition. See, e.g., U.S. Pat. No. 10,646,644 B2 to Leonhardt et al. (May 12, 2020) for “Stimulator, Pump & Composition” and U.S. Pat. No. 10,960,206 B2 to Leonhardt et al. (Mar. 30, 2021) for “Bioelectric Stimulator,” the contents of each of which are incorporated herein by this reference.

Exosomes represent a specific subset of secreted membrane vesicles, which are relatively homogeneous in size (30-100 nm). Exosomes have been proposed to differ from other membrane vesicles by its size, density, and specific composition of lipids, proteins, and nucleic acids, which reflect its endocytic origin.

Exosomes are formed in endosomal vesicles called multivesicular endosomes (MVEs) or multivesicular bodies, which originate by direct budding of the plasma membrane into early endosomes. The generation of exosomes to form MVEs involves the lateral segregation of cargo at the delimiting membrane of an endosome and inward budding and pinching of vesicles into the endosomal lumen. Because exosomes originate by two successive invaginations from the plasma membrane, its membrane orientation is similar to the plasma membrane. Exosomes from many cell types may contain similar surface proteins as the cell from which it is derived. Membrane proteins that are known to cluster into microdomains at the plasma membrane or at endosomes, such as tetraspanins (CD63, CD81, CD82), often are also enriched in EVs. It is also thought that endosomal sorting complex responsible for transport system and tetraspanins, which are highly enriched in MVEs, play a role in exosome production. How cytosolic constituents are recruited into exosomes is unclear but may involve the association of exosomal membrane proteins with chaperones, such as HSC70, that are found in exosomes from most cell types. MVEs are also sites of miRNA-loaded RNA-induced silencing complex accumulation, and the fact that exosome-like vesicles are considerably enriched in GW182 and AGO2 implicates the functional roles of these proteins in RNA sorting to exosomes. Exosomes are released to the extracellular fluid by fusion of MVE to the plasma membrane of a cell, resulting in bursts of exosome secretion. Several Rab GTPases such as Rab 27a and Rab27b, Rab11 and Rab35, all seem to be involved in exosomes release.

Repeat doses of the bioelectric stimulation and/or composition are also preferred. See, e.g., Gavira et al. “Repeated implantation of skeletal myoblast in a swine model of chronic myocardial infarction,” Eur Heart 31(8): 1013-1021. doi: 10.1093/eurheartj/ehp342 (2010), the contents of which are incorporated herein by this reference.

For heart muscle regeneration, immature myoblasts and cardiac-derived progenitors cells as well as endothelial progenitor cells (EPCs) may be included in the composition.

Typical subjects to be treated are mammals such as humans.

Stem cells may be obtained using a same-day stem cell process, which takes about 60 minutes. In such a process, first, one obtains tissue sample from the subject. Then a fat sample is processed using commercially available equipment and kits. This tissue is combined with reagent centrifuge and platelet rich fibrin. The stromal vascular fraction (“SVF”) is washed and filtered. Stem cells are re-suspended in saline or platelet rich plasma (“PRP”) and injected into the subject. The process may be repeated as needed or desired.

The SVF of adipose tissue is a source of pre-adipocytes, mesenchymal stem cells (MSC), endothelial progenitor cell, T cells, B cells, mast cells as well as adipose tissue macrophages.

A preferred composition for administration includes adipose-derived cells (or bone marrow-derived MSCs or any pluripotent stem cell, such as iPS cells) and growth factor mix which should include SDF-1, IGF-1, EGF, HGF, PDGF, VEGF, eNOS, activin A, activin B, follistatin, and tropoelastin plus selected exosomes (miR-146a, miR-294, mES-Exo) plus selected alkaloids (harmine and tetrahydroharmine) plus selected anti-inflammatory factors plus nutrient hydrogel (IGF-1, SDF-1, HGF plus FGF) plus skin matrix. Also, preferably included are amniotic fluid, placenta, or cord blood when available.

The concentration of cells in the compositions is preferably about 50,000,000 cells/ml. The amniotic fluid is preferably as described in Pierce et al. “Collection and characterization of amniotic fluid from scheduled C-section deliveries,” Cell Tissue Bank, DOI 10.1007/s10561-016-9572-7 (Springer, 2012) and is available from Irvine Scientific.

The invention is further described with the aid of the following illustrative Example.

### EXAMPLES

### Example—Controlling Expression and/or Release of S100

Purpose: The purpose of this study was to research the effects of the application of bioelectric signals on expression of S100A1 protein in porcine heart tissue, measured using RT-qPCR.

Target Proteins: The S100 protein family, composed of 25 members, is involved in a wide variety of cell types and tissues. In cardiac tissue, S100A plays a critical role in systolic calcium levels, diastolic calcium regulation, and arteriogenesis. Moreover, S100A1 normalizes cardiac function after myocardial infraction and is a critical for an adrenergic stimulation. The protein has an important role in myofilament sliding, myofilament calcium sensibility, apoptosis, and cardiac remodeling.

Bioelectric/Electrical Signals: Porcine cardiac tissue was stimulated for 30 minutes using a square, biphasic waveform at 50% duty. Frequency and signal amplitudes were fixed and set from 5 Hz to 2,500 Hz and 1.0 V, respectively.

Methods: Porcine cardiac tissue was obtained from the right ventricle and small 2-3 mm² pieces were cut with a razor blade. These pieces were set at the middle of each well plate and covered with DMEM complete media (FBS, Pen-Strep and Non-Essential amino acids). The tissue pieces were stimulated using frequencies ranging from 5 Hz to 2,500 Hz at 1 V for 30 minutes using a constant voltage waveform generator (RIGOL). Gene expression was analyzed by extracting mRNA from the tissue using a rotor stator and applying RT-qPCR assessment to quantify s100A mRNA expression, normalized to GAPDH.

Conclusions: The main effect of frequency on porcine cardiac tissue was an increase in S100A expression at 10 Hz, 75 Hz, 250 Hz, and 500 Hz. The one-sample T-Test failed to document statistical changes in S100A expression with certain frequencies, due to the power applied as only 2 samples were run per frequency. However, a general increase in expression was found with bioelectric stimulation.

One sample T-tests were as follows:

One-Sample T-Test (Frequency, Volts)

| | | | | | | | | | | P. Value. |
|---|---|---|---|---|---|---|---|---|---|---|
| ## | Frequency | Volts | N | Fold | sd | se | ci | TTest | P. Value | Adj Sig |
| ## 2 | 5 | 1 | 2 | 1.45 | 0.26 | 0.18 | 2.29 | 2.500 | 0.061 | 0.125 |
| ## 3 | 10 | 1 | 2 | 2.22 | 0.81 | 0.57 | 7.30 | 2.140 | 0.070 | 0.125 |
| ## 4 | 25 | 1 | 2 | 1.35 | 0.32 | 0.22 | 2.85 | 1.591 | 0.089 | 0.125 |
| ## 5 | 50 | 1 | 2 | 2.25 | 1.02 | 0.72 | 9.14 | 1.736 | 0.083 | 0.125 |
| ## 6 | 75 | 1 | 2 | 2.64 | 1.01 | 0.71 | 9.03 | 2.310 | 0.065 | 0.125 |
| ## 7 | 100 | 1 | 2 | 1.51 | 0.47 | 0.33 | 4.19 | 1.545 | 0.091 | 0.125 |
| ## 8 | 250 | 1 | 2 | 1.89 | 0.14 | 0.10 | 1.29 | 8.900 | 0.018 | 0.125 |
| ## 9 | 500 | 1 | 2 | 2.30 | 0.32 | 0.23 | 2.86 | 5.652 | 0.028 | 0.125 |
| ## 10 | 750 | 1 | 2 | 1.51 | 0.64 | 0.46 | 5.79 | 1.109 | 0.117 | 0.131 |
| ## 11 | 1000 | 1 | 2 | 1.04 | 0.15 | 0.11 | 1.34 | 0.364 | 0.194 | 0.194 |
| ## 12 | 2500 | 1 | 2 | 1.66 | 0.86 | 0.61 | 7.72 | 1.082 | 0.119 | 0.131 |

### REFERENCES

(The contents of the entirety of each of which is incorporated herein by this reference.)

“Cocktail of Factors from Fat-derived Stem Cells Shows Promise for Critical Limb Ischemia,” world wide web at sciencenewsline.com/news/2016012204520017.html (Jan. 22, 2016).

Gavira et al. “Repeated implantation of skeletal myoblast in a swine model of chronic myocardial infarction,” Eur Heart J, 31(8): 1013-1021. doi: 10.1093/eurheartj/ehp342 (2010).

Pierce et al. “Collection and characterization of amniotic fluid from scheduled C-section deliveries,” Cell Tissue Bank, DOI 10.1007/s10561-016-9572-7 (Springer, 2012).

Procházka et al. “Therapeutic Potential of Adipose-Derived Therapeutic Factor Concentrate for Treating Critical Limb Ischemia,” Cell Transplantation, 25(9), pp. 1623-1633(11) (2016).

Wang, X. J., & Wang, M. “The S100 protein family and its application in cardiac diseases” World Journal of Emergency Medicine, 1(3), 165-168 (2010).

Winters et al. “Evaluation of Multiple Biological Therapies for Ischemic Cardiac Disease” Cell Transplantation, Vol. 25, pp. 1591-1607 (2016).

Xia et al. “S100 Proteins As an Important Regulator of Macrophage Inflammation” Front. Immunol., (Jan. 5, 2018); https://doi.org/10.3389/fimmu.2017.01908.

U.S. Pat. No. 10,646,644 B2 to Leonhardt et al. (May 12, 2020) for “Stimulator, Pump & Composition.”

U.S. Pat. No. 10,960,206 B2 to Leonhardt et al. (Mar. 30, 2021) for “Bioelectric Stimulator.”

U.S. Patent Application Publication US 2020-0289826 A1 to Leonhardt et al. (Sep. 17, 2020) for “Klotho Modulation.”

What is claimed is:

1. A bioelectric stimulator programmed to produce at least one bioelectric signal that modulates S100 in a cellular target tissue when the at least one bioelectric signal is applied to the target cellular tissue.

2. The bioelectric stimulator of claim 1, wherein the bioelectric stimulator is programmed to produce a bioelectric signal that upregulates expression of S100 in the target cellular tissue as may be assessed by using RT-qPCR.

3. The bioelectric stimulator of claim 2, wherein the at least one bioelectric signal is selected from the group consisting of 10 Hz square, biphasic waveform at 50% duty, 75 Hz square, biphasic waveform at 50% duty, 250 Hz square, biphasic waveform at 50% duty, 500 Hz square, biphasic waveform at 50% duty, and a combination of any thereof.

4. The bioelectric stimulator of claim 3, wherein the bioelectric signal(s) comprise(s) 10 Hz square, biphasic waveform at 50% duty.

5. The bioelectric stimulator of claim 3, wherein the bioelectric signal(s) comprise(s) 75 Hz square, biphasic waveform at 50% duty.

6. The bioelectric stimulator of claim 3, wherein the bioelectric signal(s) comprise(s) 250 Hz square, biphasic waveform at 50% duty.

7. The bioelectric stimulator of claim 3, wherein the bioelectric signal signal(s) comprise(s) 500 Hz square, biphasic waveform at 50% duty.

8. The bioelectric stimulator of claim 2, wherein the at least one bioelectric signal is from about 250 Hz square, biphasic waveform to about 500 Hz square, biphasic waveform.

9. The bioelectric stimulator of claim 1, which is further programmed to produce a bioelectric signal or signals that modulate(s) expression in cellular tissue of at least one other protein selected from the group consisting of klotho, LIM muscle, follistatin, stromal cell-derived factor 1 (SDF1), platelet-derived growth factor (PDGF), epidermal growth factor (EGF), hepatocyte growth factor (HGF), Insulin-like growth factor 1 (IGF-1), and any combination thereof.

10. A method of using the bioelectric stimulator of claim 1 to stimulate tissue of a subject to upregulate expression of S100A in the subject, the method comprising:
stimulating target cellular tissue of the subject by applying at least one bioelectric signal produced by the bioelectric stimulator to the tissue to upregulate expression of S100A in the subject.

11. The method according to claim 10, further comprising:
delivering to the tissue a composition comprising human umbilical cord sub-epithelial cell-derived stem cells (hUC-SECs), human bone marrow-derived mesenchymal stem cells (hBM-MSCs), and human induced pluripotent stem cell-derived cardiomyocytes (hiPSC-CMs).

12. The method according to claim 10, wherein the tissue is the subject's muscle tissue.

13. The method according to claim 12, further comprising actuating the bioelectric stimulator to produce bioelectric signals that modulate expression of at least one other protein selected from the group consisting of klotho, LIM muscle, follistatin, stromal cell-derived factor 1 (SDF1), platelet-derived growth factor (PDGF), epidermal growth factor (EGF), hepatocyte growth factor (HGF), and any combination thereof.

14. The method according to claim 10, wherein the subject has been diagnosed as suffering from bladder, heart, and/or nerve tissue damage.

15. A method of treating a cell, the method comprising:
stimulating the cell to express and/or release a S100 peptide by applying at least one bioelectric
signal to the cell, wherein the at least one bioelectric signal is selected from the group
consisting of 10 Hz square, biphasic waveform at 50% duty, 75 Hz square, biphasic
waveform at 50% duty, 250 Hz square, biphasic waveform at 50% duty, 500 Hz square,
biphasic waveform at 50% duty, and a combination of any thereof.

16. The method according to claim 15, wherein the bioelectric signal(s) comprise(s) a 75 Hz square, biphasic waveform at 50% duty.

17. The method according to claim 15, wherein the bioelectric signal(s) comprise(s) a 250 Hz square, biphasic waveform at 50% duty.

18. The method according to claim 15, wherein the bioelectric signal signal(s) comprise(s) a 500 Hz square, biphasic waveform at 50% duty.

19. The method according to claim 15, wherein the cell is comprised within a subject.

20. The method according to claim 19, wherein the tissue is cell is a bladder, heart, or nerve cell.
